(19) United States
(12) Patent Application Publication
(10) Pub. No.: US 2006/0069270 A1
(43) Pub. Date: Mar. 30, 2006

(54) Process for the preparation of 1,3,5-trisubstituted pyrazoles via [3+2] cycloaddition

(76) Inventors: Rafael Shapiro, Wilmington, DE (US); Lucius T. Rossano, West Windsor, NJ (US); Karen L. Tenhuisen, Randolph, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/220,017

(22) Filed: Sep. 6, 2005

Related U.S. Application Data
(60) Provisional application No. 60/613,472, filed on Sep. 27, 2004.

(51) Int. Cl. C07D 231/14

(52) U.S. Cl. 548/374.1

(57) ABSTRACT

A process for the preparation of 1,3,5-trisubstituted pyrazole compounds, which are useful intermediates in the synthesis of factor Xa inhibitors.

### RELATED APPLICATIONS

This application claims priority benefit under Title 35 § 119(e) of U.S. Provisional Application No. 60/613,472, filed Sep. 27, 2004, the contents of which are herein incorporated by reference.

### FIELD OF THE INVENTION

This invention relates to a process for preparing a 1,3,5-trisubstituted pyrazole compound, an intermediate useful in the synthesis of factor Xa inhibitors.

### BACKGROUND OF THE INVENTION

Factor Xa inhibitors, such as a compound of formula I, 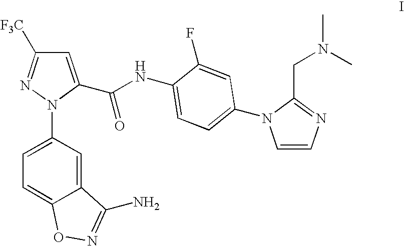
have been investigated as potential drug candidates for the treatment and prevention of thromboembolic disorders. See U.S. Pat. No. 6,339,099. Accordingly, there is a need to produce a large amount of these compounds for clinical studies.

WO98/57951 discloses the preparation of the compound of formula I as in Scheme 1. A pyrazolecarboxylic acid of formula 1-1 and aniline of formula 1-2 can be coupled to give an amide of formula 1-3, which can be further converted to the compound of formula I by reacting with acetone oxime in the presence of a base, followed by cyclization in the presence of an acid.

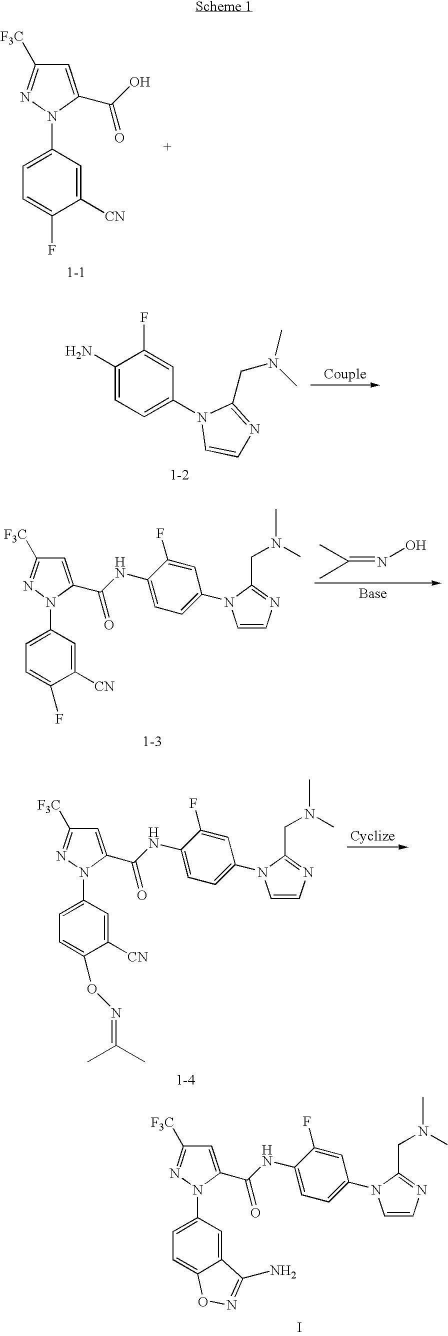

WO01/29006 describes a method for preparing a pyrazolecarboxylic acid of formula VII as shown in Scheme 2. In Scheme 2, D is 2-cyanophenyl, 2-(Pg-NHCH₂)phenyl, 2-(aminomethyl)phenyl, 3-cyanophenyl, 3-(Pg-NHCH₂)phenyl, 3-(aminomethyl)phenyl, 3-cyano-4-fluorophenyl, or (3-amino)benz[d]isoxazol-6-yl, wherein Pg is an amine protecting group; X is Cl, Br, OMs, OSO₂Ph, or OTs; R′ is H, Me, Et, or n-propyl. Thus, a hydrazine of formula II can react with an acylating reagent, such as trifluoroacetic anhydride, to give a compound of formula III. The compound of formula III can further react with a suitable reagent to produce a compound of formula IV. For example, Compound III can react with CCl₄ in the presence of PPh₃, or alternatively MsCl in the presence of a base such as Et₃N, to give compound IV wherein X is Cl. Likewise, Compound III can react with CBr₄ in the presence of PPh₃ to generate compound IV wherein X is Br. Similarly, Compound III can react with a suitable sulfonyl halide reagent, such as methanesulfonyl chloride, benzenesulfonyl chloride and toluenesulfonyl chloride, in the presence of a base such as trialkylamine (e.g., triethylamine) or diisopropylethylamine, to afford Compound IV wherein X is OMs, OSO₂Ph, or OTs. Additionally, Compound III can react with a sulfonic anhydride agent selected from methanesulfonic anhydride, benzenesulfonic anhydride and toluenesulfonic anhydride, in the presence of a base, preferably pyridine. Compound IV can react with an alkene derivative of formula Vₐ in the presence of a base to produce a pyrazoline of formula VIₐ through [3+2] cycloaddition (here, Compound IV is converted in situ to a nitrileimine, a 1,3-dipole). Finally, a compound of formula VII can be generated by aromatizing Compound VIₐ to a corresponding pyrazole, followed by hydrolysis if R′ is not hydrogen. The aromatization can be achieved with oxidation with oxygen in air under basic conditions, or eletrophilic chlorination with N-chlorosuccinimide (NCS) followed by in situ elimination of hydrogen chloride.

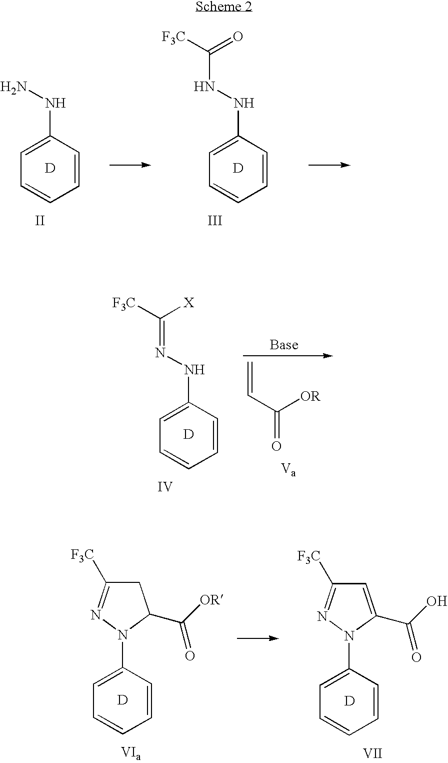

Since the pyrazole carboxylic acid of formula VII is a key intermediate in the synthesis of factor Xa inhibitors like the compound of formula I, there remains a need to find a more efficient method suitable for commercial manufacturing of Compound VII.

The entire disclosure of each of the foregoing patent applications, patents, and patent publications is incorporated herein by reference.

### SUMMARY OF THE INVENTION

The present invention relates to a process for preparing a compound of formula VI, 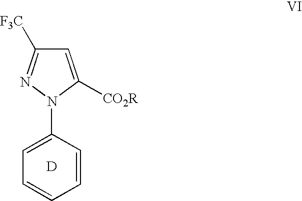
wherein D is 2-cyanophenyl, 2-(Pg-NHCH₂)phenyl, 2-(aminomethyl)phenyl, 3-cyanophenyl, 3-(Pg-NHCH₂)phenyl, 3-(aminomethyl)phenyl, 3-cyano-4-fluorophenyl, or (3-amino)benz[d]isoxazol-6-yl, in which Pg is an amine protecting group; and R is C₁-C₄ alkyl,
comprising reacting a compound of formula IV, 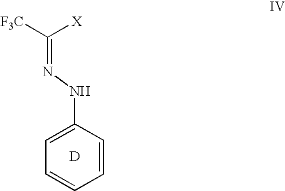
wherein X is Cl, Br, OMs, OSO₂Ph, or OTs,
with a compound of formula V_{b}, 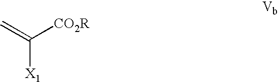
wherein X₁ is Br or Cl, and R is defined same as hereinabove,
in the presence of a base.

### DETAILED DESCRIPTION OF THE INVENTION

### Definitions

The term “alkyl” or “alk” refers to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “C₁-C₄ alkyl” refers to a straight or branched chain alkane (hydrocarbon) radical containing from 1 to 4 carbon atoms, such as methyl, ethyl, propyl, isopropyl, n-butyl, t-butyl, and isobutyl.

The terms “halogen” or “halo” refer to chlorine, bromine, fluorine or iodine.

When a functional group is termed “protected”, this means that the group is in modified form to mitigate, especially preclude, undesired side reactions at the protected site. The term “amino protecting group” means suitable protecting groups for amines. The methods and compounds described herein include, without limitation, those described in standard textbooks, such as Greene, T. W. et al., Protective Groups in Organic Synthesis, Wiley, N.Y. (1999), which is incorporated herein by reference.

The compounds of present invention may form salts which are also within the scope of this invention. Reference to compounds of the formula I through VII herein is understood to include reference to salts thereof, unless otherwise indicated. The term “salt(s)”, as employed herein, denotes acidic and/or basic salts formed with inorganic and/or organic acids and bases. In addition, when a compound contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds may be formed, for example, by reacting those compounds with an amount of acid or base, such as an equivalent amount, in a medium such as one in which the salt precipitates or in an aqueous medium followed by lyophilization.

The compounds of present invention may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of present invention may also form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield compounds of the formula I through V, or a salt and/or solvate thereof. Solvates of the compounds of formula I through V include, for example, hydrates.

Compounds of the formula I through VII, and salts thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specifications, groups and substituents thereof may be chosen to provide stable moieties and compounds.

### Methods of Preparation

The methods for preparing compounds of formula VI and VII are illustrated in the following scheme. Solvents, temperatures, pressures, and other reaction conditions may readily be selected by one of ordinary skill in the art. Starting materials are commercially available or readily prepared by one of ordinary skill in the art.

In Scheme 3, D is 2-cyanophenyl, 2-(Pg-NHCH₂)phenyl, 2-(aminomethyl)phenyl, 3-cyanophenyl, 3-(Pg-NHCH₂)phenyl, 3-(aminomethyl)phenyl, 3-cyano-4-fluorophenyl, or (3-amino)benz[d]isoxazol-6-yl, in which Pg is an amine protecting group; R is C₁-C₄ alkyl; X is Cl, Br, OMs, OSO₂Ph, or OTs; and X₁ is Br or Cl. Preferably, D is 3-cyano-4-fluorophenyl; R is Me or Et; X is Cl; and X₁ is Cl.

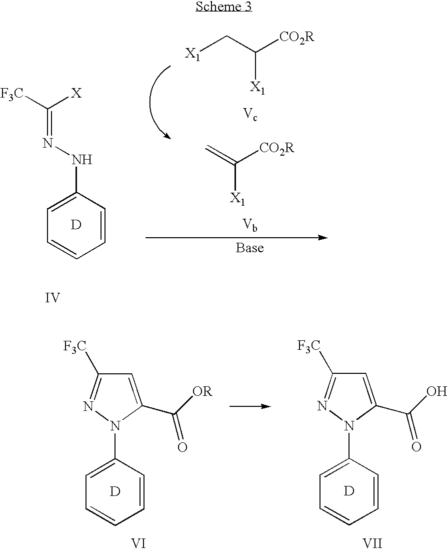

According to Scheme 3, the compound of formula IV (which can be prepared in accordance with Scheme 2) can react with an alkene of formula V_{b} in the presence of a base, such as a tertiary amine, to give a compound of formula VI via [3+2] cycloaddition. The alkene of formula V_{b} can be generated in situ by contacting a compound of formula V_{c} with a base, such as a tertiary amine base. The compound of formula VI can be further hydrolyzed to afford a pyrazole carboxylic acid of formula VII upon the treatment of a base, such as LiOH, NaOH, or KOH, in an organic solvent, such as THF.

### Abbreviations

Aq. aqueous
DIPEA diisopropylethylamine
HOAc: acetic acid
IPA: isopropyl alcohol
MeOH: methyl alcohol
OMs: OSO₂CH₃
OSO₂Ph: OSO₂phenyl
OTs: OSO₂-phenyl-4-methyl
RTD: resistance temperature detector
THF: tetrahydrofuran

The features and advantages of the present invention are more fully shown by the following examples which are provided for purposes of illustration, and are not to be construed as limiting the invention in anyway.

### EXAMPLES

HPLC Condition:

Column: Agilent Zorbax Eclipse XDB, C-8, 250X4.6 mm, 5 um;
Phase A: 1.9 g of NH₄OAc in 1 L 10% MeOH;
Phase B: 1.9 g of NH₄OAc in 1 L 90% MeOH;
Gradient: initial B 40%, final B 100%, time 30 min.

This HPLC condition was used for the following examples.

### Example 1

Preparation of Compound 3:

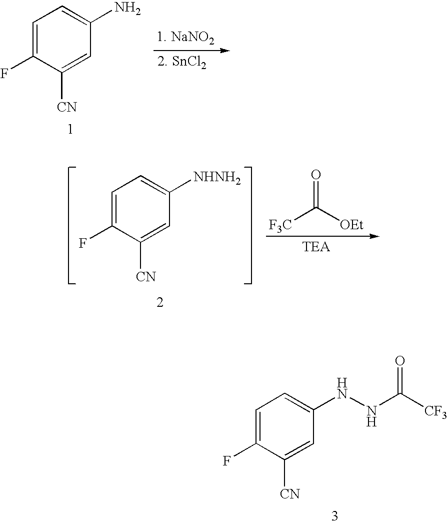

Diazonium salt formation: To a jacketed 4 L Chemglass reactor fitted with a baffle, addition funnel, RTD, nitrogen inlet, overhead stirrer, and condenser was charged with Compound 1 (375 g, 2.75 mol) and acetic acid (1108 g, 18.46 mol). The solution was stirred under nitrogen while gradually charging water (1125 mL). The jacket setpoint was then set to −25° C. and 37% aq. HCl (1375 mL, 16.5 mol) was added gradually. While this mixture was cooling, a solution of sodium nitrite (209.1 g, 3.03 mol) in 25 mL of water was prepared. After the mixture was cooled to −20° C., the sodium nitrite solution was added dropwise, keeping the temperature below −10° C.

Stannous chloride mixture preparation: While the reaction to form the diazonium salt was occurring, anhydrous tin (II) chloride (1149 g, 189.6 mol) was charged to a jacketed 20 L Chemglass reactor fitted with a baffle, RTD, nitrogen inlet, overhead stirrer, and condenser. Water (1100 mL) and then 37% HCl (815 mL) were added to form thick slurry. The jacket setpoint was then set to −25° C. and the mixture was cooled to about −25° C.

Stannous chloride reduction: After about 2 h at −10° C., HPLC evidenced less than 1% Compound 1 in the diazonium reaction and the resulting mixture was vacuum transferred very slowly into the stannous chloride mixture maintaining the temperature below about −5° C. The reaction mixture was allowed to stir overnight and the reaction completion was indicated by the disappearance of the diazonium hydrochloride peak by HPLC analysis. Prior to quenching, the reaction was diluted with water (1875 mL) and treated with 50% sodium hydroxide dropwise via addition funnel while maintaining the batch temperature below 0° C. After adjusting the pH value of the reaction mixture to about 5 using 50% sodium hydroxide, tetrahydofuran was added (5625 mL). The basification was then continued until the pH value reached to about 13-13.5. The basified mixture was then agitated for about 1 h and then held without agitation for about 0.5 h. After separating the phases, the organic layer was washed with saturated brine (1.5 L).

Trifluoroacetylation: After separating the phases, the organic phase was azeotropically dried at atmospheric pressure to remove water. The resulting dark brown mixture was treated with triethylamine (395 g, 3.9 mol) and ethyl trifluoroacetate (548 g, 3.9 mol) and heated at 40° C. overnight (about 16 h). Additional charges of triethylamine (112 g, 1.10 mol) and ethyl trifluoroacetate (156 g, 1.10 mol) were added and the reaction mixture was reheated to 50° C. to drive the reaction to completion, which was evidenced by the disappearance of Compound 2. After cooling the reaction mixture to about 20° C., the reaction was diluted with IPA (940 mL), treated with acetic acid (298 mL, 4.96 mol) and diluted with water via dropwise addition of water (3450 mL) over about 1 h. The resulting mixture was cooled to 0-5° C. and held for about 16 h. The red-brown solids which precipitated were filtered, washed with 10% IPA/water (1200 mL), and dried at 50° C. to give Compound 3 as red-orange solids (488.7 g, 73% yield).

Purification via Zeta Pad treatment: The red-orange solids (450 g) isolated above were dissolved in IPA (4.50 L) and purified by eluting over a Cuno Zeta pad (53 sp, 6 cell, 8″ diameter) with IPA (16 L) at 100 mL per minute. The filtrates were combined and concentrated to about 900 mL. When crystals started to form, the mixture was cooled to 0-5° C. for about 2 h, filtered, and washed with 50% IPA/water (200 mL) to give Compound 3 as white crystals (246 g, 98.8 wt %, 99.2 AP, and 36.2% yield). A second crop was crystallized from the mother liquor by concentrating and precipitating with water to give, after drying, an off-white solid (106 g, 15.6% yield, 97.7 wt %). Total isolated yield for the two crops is 51.8% (352 g). Total recovery for Cuno process from the crude red-brown solid is 72%.

### Example 2

Preparation of Compound 5:

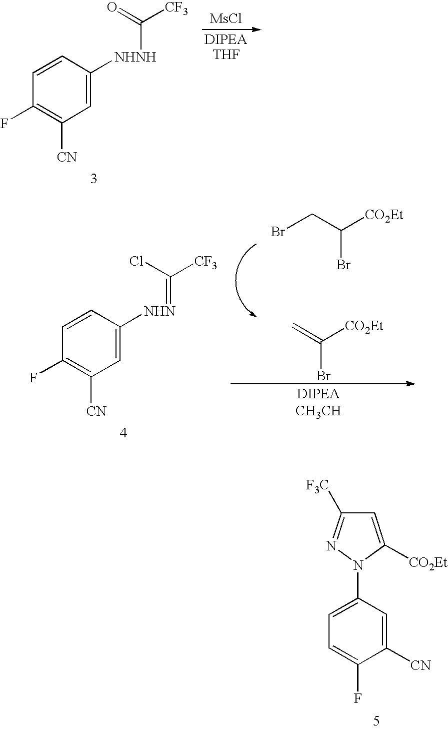

A jacketed reaction flask equipped with stirrer was charged with Compound 3 (1.5 kg) and THF (22.53 L) at ambient temperature, followed by DIPEA (1.59 L). The reaction mixture was cooled to 0-5° C. and MsCl (0.49 L) was added dropwise from an addition funnel, and the reaction temperature was kept at about 5° C. After the addition was completed, the reaction mixture was allowed to warm to ambient temperature. When HPLC indicated the reaction was complete with the disappearance of Compound 3 and the formation of Compound 4, the THF used in the reaction was distilled off (about 90%) under reduced pressure, and acetonitrile (15.18 L) was charged to the reaction mixture, followed by DIPEA (4.76 L) and ethyl 2,3-dibromopropionate (1.5 L). With agitation, the reaction mixture was heated to about 45° C. and maintained at this temperature until HPLC indicated the reaction was complete. The reaction mixture was cooled to ambient temperature, charged with MeOH (18.22 L), and followed by adding water (26.3 L) dropwise with agitation, to give a slurry. The slurry was cooled to about 5° C. and continued with agitation for about 2 hours. The slurry was filtered, washed with water (2.21 L), dried under vacuum to afford Compound 5 as a pale brown solid. Typical yield was about 70%. HPLC Retention time: 23.11 min. ¹H NMR (300 MHz, DMSO-d₆): δ 8.37 (br d, J=6 Hz, 1H); δ 8.08 (br m, 1H); δ 7.73 (br dd, J=9 Hz, 0.5 Hz, 1H); δ 7.65 (s, 1H); δ 4.22 (q, J=6 Hz, 2H); δ 1.18 (t, J=6 Hz, 3H).

### Example 3

Formation of Compound 6:

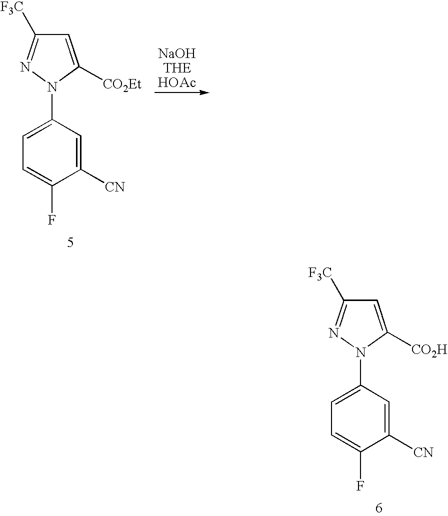

Compound 5 (187.5 g) was dissolved in THF (940 mL) with agitation and cooled to 5° C. 2N NaOH (319 mL) was charged dropwise, and the reaction temperature was kept at 5-10° C., until the reaction was complete as indicated by HPLC. Next, HOAc (1.275 L) was added to the reaction mixture and THF was removed under reduced pressure at about 45-50° C., and then water (2.6 L) was added dropwise to precipitate the product. The slurry was cooled to 5° C., held for about 1 hour, filtered, washed with water (2.65 L), and dried under vacuum. The yield was about 147 g, 87%. HPLC Retention time: 10.88 min. ¹H NMR (300 MHz, DMSO-d₆): δ 14.01 (br s, 1H, OH); δ 8.35 (dd, J=3 Hz, 0 Hz, 1H); δ 8.06 (ddd, J=9 Hz, 3 Hz, 0.5 Hz, 1H); δ 7.71 (dd, J=9 Hz, 3 Hz, 0.5 Hz, 1H); δ 7.56 (s, 1H).

What is claimed is:

1. A process for preparing a compound of formula VI, or a pharmaceutically acceptably salt thereof, 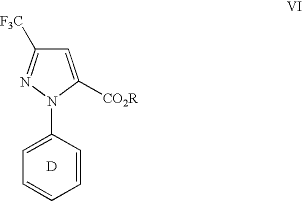
wherein D is 2-cyanophenyl, 2-(Pg-NHCH₂)phenyl, 2-(aminomethyl)phenyl, 3-cyanophenyl, 3-(Pg-NHCH₂)phenyl, 3-(aminomethyl)phenyl, 3-cyano-4-fluorophenyl, or (3-amino)benz[d]isoxazol-6-yl, in which Pg is an amine protecting group; and R is C₁-C₄ alkyl,
comprising reacting a compound of formula IV, 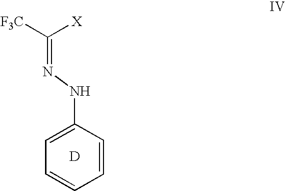
wherein X is Cl, Br, OMs, OSO₂Ph, or OTs,
with a compound of formula Vb in the presence of a base, 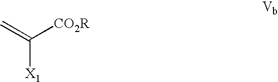
wherein X₁ is Br or Cl, and R is defined same as hereinabove.

2. The process of claim 1, wherein the compound of formula V_{b} is generated in situ be contacting a compound of formula V_{c} with a base, 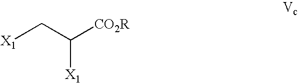
in which X₁ and R are defined same as in claim 1.

3. The process of claim 1, wherein the compound of formula IV is generated by reacting a compound of formula III, 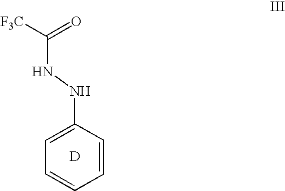
wherein D is defined same as in claim 1,
with a tetra-halogen carbon reagent selected from CCl₄ and CBr₄, in the presence of PPh₃;
or a sulfonyl halide reagent selected from methanesulfonyl chloride, benzenesulfonyl chloride and toluenesulfonyl chloride, in the presence of a base;
or a sulfonic anhydride reagent selected from methanesulfonic anhydride, benzenesulfonic anhydride and toluenesulfonic anhydride, in the presence of a base.

4. The process of claim 1, wherein D is 3-cyano-4-fluorophenyl; R is Me or Et; X is Cl; and X₁ is Br.

5. The process of claim 4, wherein the compound of formula V_{b} is generated in situ be contacting a compound of formula V_{c} with a base, 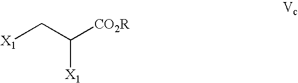
in which X₁ and R are defined same as in claim 4.

6. The process of claim 4, wherein the compound of formula IV is generated by reacting a compound of formula III, 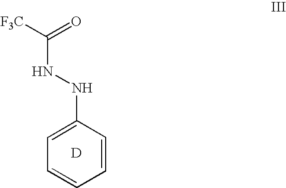
wherein D is defined same as in claim 4,
with a sulfonyl halide reagent selected from methanesulfonyl chloride, benzenesulfonyl chloride and toluenesulfonyl chloride, in the presence of a tertiary amine base.

7. The process of claim 6, wherein the tertiary amine base is triethylamine, diisopropylethylamine, or N-methylmorpholine.

8. The process of claim 1, further comprising hydrolyzing the compound of formula VI to give a compound of formula VII, or a pharmaceutically acceptably salt thereof.

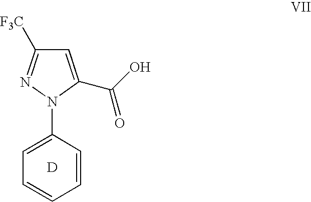

9. The process of claim 8, wherein D is 3-cyano-4-fluorophenyl.
